(19) United States
(12) Patent Application Publication
(10) Pub. No.: US 2024/0423717 A1
(43) Pub. Date: Dec. 26, 2024

(54) DEVICE CONNECTING A SURGICAL LASER LIGHT APPLICATION FIBER TO A LASER, AND LASER APPARATUS COMPRISING A SURGICAL LASER LIGHT APPLICATION FIBER

(71) Applicant: Futonics Laser GmbH, Katlenburg-Lindau (DE)

(72) Inventor: Peter Fuhrberg, Göttingen (DE)

(73) Assignee: Futonics Laser GmbH, Katlenburg-Lindau (DE)

(21) Appl. No.: 18/827,780

(22) Filed: Sep. 8, 2024

Related U.S. Application Data
(63) Continuation of application No. PCT/EP2023/055973, filed on Mar. 9, 2023.

(30) Foreign Application Priority Data
Mar. 9, 2022 (DE) 10 2022 105 499.3

(51) Int. Cl. A61B 18/26; A61B 18/00; A61B 18/20; A61B 18/22; G02B 6/42

(52) U.S. Cl. CPC A61B 18/26; G02B 6/4206; G02B 6/4228; A61B 2018/00172; A61B 2018/2025; A61B 2018/2205

(57) ABSTRACT

A laser apparatus comprises a laser outputting laser light at an output location in a mean propagation direction, a surgical laser light application fiber having a fiber axis and a light entry cross section, and a device for connecting the fiber to the laser. The device comprises laser and fiber ports connecting the laser and the fiber to the device, and focusing optics arranged between the laser and fiber ports and coupling a part of the laser light output by the laser at the output location through the light entry cross section into the fiber. The focusing optics focus the laser light with a full convergence angle of not more than 2° onto the light entry cross section; and the focusing optics couple the laser light at an angle between the mean propagation direction and the fiber axis in a range from 2° to 10° into the fiber.

### CROSS REFERENCE TO RELATED APPLICATIONS

This application is a continuation of International Application PCT/EP2023/055973 with an international filing date of Mar. 9, 2023 and claiming priority to co-pending German Patent Application No. DE 10 2022 105 499.3 entitled “Vorrichtung zum Anschließen einer Applikationsfaser zum Applizieren von Laserlicht für chirurgische Anwendungen an einen Laser und Laserapparatur mit einem Laser und einer solchen Applikationsfaser” and filed on Mar. 9, 2022, the disclosures of which are hereby incorporated herein by reference in their entirety.

### FIELD OF THE INVENTION

The invention relates to a device for connecting a surgical laser light application fiber to a laser. Further, the invention relates to a laser apparatus comprising a surgical laser light application fiber. Such a laser apparatus may, for example, be used for comminuting water-containing stones, like, for example, kidney stones, in body cavities. In order to, for example, comminute water-containing stones in body cavities by means of laser light, the laser light provided by a laser is guided to the location of its application with an application fiber, i.e. an optical fiber. In the example selected, the laser light exiting out of the application fiber acts in the body cavity upon the respective water-containing stone to heat it up locally in such a way that it disintegrates into smaller parts. In this application, the application fiber is stressed and contaminated, particularly at its distal fiber end out of which the laser light exits. After a certain period of use, the application fiber is used up to such an extent that it has to be replaced. Thus, devices for connecting an application fiber to a laser, which allow for a quick replacement of the application fiber, are usual.

### BACKGROUND OF THE INVENTION

For comminuting kidney stones in the sense of fragmenting, the use of a Holmium:YAG laser is known whose laser light is guided directly to the respective kidney stone via an application fiber inserted via the ureter. The laser light of the Holmium:YAG laser of a wavelength of 2,100 nm exiting out of the application fiber heats up the kidney stone, as a whole or locally, such that it is fragmented by vaporizing water components.

In order to securely achieve this, the use of Holmium:YAG lasers having a maximum peak power of 15 kW is usual. Such Holmium:YAG lasers, particularly, those which achieve a pulse repetition rate of more than 30 Hz, are expensive to manufacture. Further, the risk potential of the laser light existing at this maximum power is high, if the laser light not only hits on the kidney stone as planned but also on neighboring kidney tissue.

In fragmenting kidney stones with laser light from a Holmium:YAG laser comprising a laser head, the laser light is typically applied in pulses of a pulse energy higher than one joule and a pulse frequency lower than 30 Hz. Holmium:YAG lasers having up to four laser heads and an overall average power of 120 W are known for fragmenting kidney stones. The reported highest fragmentation rates achieved in practice are typically about 6.6 mg kidney stones per second at an average power of 120 W.

For comminuting kidney stones in the sense of pulverizing, the use of a thulium fiber laser is proposed. Its laser light of a wavelength of 1,940 nm is by a factor of 2 better absorbed than laser light of a wavelength of 2,100 nm. Due to its essentially better beam quality, a thulium fiber laser can be used in combination with thinner and more flexible application fibers than a Holmium:YAG laser. Further, thulium fiber lasers are more compact, less expensive than multi head lasers and more durable than Holmium:YAG lasers. Herein, the by a factor of more than 10 higher energy efficiency of a thulium fiber laser is a relevant factor.

In pulverizing kidney stones with laser light from a thulium fiber laser, the laser light is typically applied in pulses of low pulse energy of about 0.1 joule at a high pulse repetition rate of typically some 100 Hz. The maximum comminuting rates achieved in practice are about 3 mg kidney stones per second.

If kidney stones shall selectively be pulverized or fragmented, both a thulium fiber laser with a connected special application fiber and a Holmium:YAG laser with a connected special application fiber are used at present.

A system for surgical endovenous laser treatment including a laser device and an application module are known from German patent DE 10 2016 118 663 B3 and United States patent application publication US 2019/0336217 A1 belonging to the same patent family. The laser device comprises a laser light source having a first laser diode element; and the application module is optically connected to the laser light source. The first laser diode element comprises a semiconductor layer made of an antimonide compound and is configured such that the laser light can be generated with a wavelength between 1,800 nm and 2,000 nm. The application module is made as a bendable catheter comprising an optical wave guide. A controller of the laser device is configured such that the laser light is emitted continuously and has a laser light power of at least 10 W at a distal end of the optical wave guide. The laser device further includes an auxiliary laser light source and a light sensor. The auxiliary laser light source and the light sensor are connected to the controller in such a way that a laser light intensity of the laser light source is adjusted depending on a specific fluorescence light signature of the tissue to be treated.

A laser welding apparatus is known from United States patent application publication US 2020/0306878 A1. The laser welding apparatus comprises a first laser device coupling a first laser beam into a first optical feed fiber, and a second laser device coupling a second laser beam into a second optical feed fiber. Further, means are provided for generating a composite laser beam from the first beam and the second laser beam exiting out of the respective optical feed fiber. The exiting first laser beam has a circular cross section and the exiting second laser beam has a ring shape which is concentric with regard to the exiting first laser beam. The means for generating the combined laser beam comprise an optical feed fiber with two concentrically arranged cores and a multi-hole capillary tube into which the optical feed fibers from the first and second laser devices enter a first side and out of which the optical feed fiber with the two cores emerges at the other side.

An apparatus and a method for varying the beam parameter product of diode lasers in laser material processing are known from German patent application publication DE 10 2020 116 268 A1 and United States patent application publication US 2021/0394304 A1 belonging to the same patent family. In laser material processing, particularly for cutting or remote welding applications, lasers with a good beam parameter product are used. However, single mode lasers in a wavelength range of 1 μm have a low cutting efficiency, if cutting of medium or thicker materials is concerned. In cutting material, a so-called mono mode beam profile only produces a very thin cut which is not broad enough to eject the molten material with thicker materials. The apparatus for laser material processing comprises laser diodes as laser sources, a focusing lens, a fiber, into which the laser light from the laser diode is coupled, wherein the beam parameter product of the fibers is higher than the beam parameter product of the incident laser light, and a substrate for generating an offset with regard to the beam axis. The method aims at increasing the coupling divergency for reducing the beam parameter product. It includes generating a laser beam by means of a diode laser, focusing the laser beam by means of a focusing lens, and coupling the laser beam into a fiber, wherein the divergence, spot size and the angle of incidence of the laser beam are varied by means of a substrate. The divergence or spot size or the angle of incidence are varied at the fiber entrance to generate a true variation of the beam parameter product within the limits of the fiber or a ring in the core. By using a fiber with a higher angle of acceptance (NA) than that one of the incident laser beam, the beam parameter product at the fiber exit can be reduced in that the divergence of the angle of incidence or the angle of incidence are increased at the input side. A donut profile is generated as the spot geometry of the laser beam exiting the fiber, if the incident divergence is much smaller than the angle of acceptance of the fiber and the spot size is smaller than the core size. The minimum beam parameter product is defined by the basic properties of the diode laser and is, thus, low enough to cut thin materials well. Due to the purposeful variation of the beam parameter product, an adaption to other materials by means of an optimized beam characteristic with regard to the beam parameter product or spot donut profile may be made.

A method of operating a laser device and a laser device comprising a laser tool whose working distance with regard to a work piece is variable within a setting range, are known from European Patent EP 1 773 534 B2 and United States patent application publication US 2007/0278194 A1 belonging to the same patent family. The laser tool has laser optics comprising a focusing and a collimating optic, and a port for an optical wave guide with a wave guide decoupling point. The focal length of the focusing optic is varied by adjusting the optical distance of the wave guide decoupling point to the collimating optic, wherein the collimating optic is moved in beam direction by means of a motor driven setting device with remote control. The setting device is connected to the controller of a multi-axial manipulator which guides the laser tool or the work piece, wherein movements of the setting device and the manipulator are linked.

There still is a need of a device for connecting an application fiber to a laser and a laser apparatus comprising a laser with an application fiber, by which the laser light is applied in a particularly efficient way in order to, for example, comminute water-containing stones in body cavities.

### SUMMARY OF THE INVENTION

The present invention relates to a device for connecting a surgical laser light application fiber to a laser, the surgical laser light application fiber having a fiber axis and a light entry cross section running crosswise to the fiber axis, and the laser outputting laser light at a light output location in a mean laser light propagation direction. The device comprises a laser port for connecting the laser to the device, and a fiber port for connecting the surgical laser light application fiber to the device. The laser port orientates the light output location and the mean laser light propagation direction of the laser light with regard to the device, and the fiber port orientates the fiber axis and the light entry cross section of the surgical laser light application fiber with regard to the device. The device further comprises a laser light beam path extending between the laser port and the fiber port, and focusing optics arranged in the laser light beam path and coupling a part of the laser light output by the laser connected to the laser port at the light output location through the light entry cross section of the surgical laser light application fiber into the surgical laser light application fiber connected to the fiber port. The focusing optics focus the part of the laser light with a full convergence angle of not more than 2° onto the light entry cross section. Further, the focusing optics couple the part of the laser light into the surgical laser light application fiber at an angle between the mean laser light propagation direction of the laser light and the fiber axis, wherein the angle is either a fixed angle in a range from 2° to 10°, or the fiber port is tiltable with regard to the focusing optics such that the angle is adjustable within a range from 0° to at least 5°.

The present invention also relates to a laser apparatus for surgical applications. The laser apparatus comprises a laser which outputs laser light with a mean laser light propagation direction at a light output location, and a surgical laser light application fiber for applying the laser light, the surgical laser light application fiber having a fiber axis and a light entry cross section running perpendicular to the fiber axis. The laser apparatus further comprises a laser light beam path extending between the laser and the surgical laser light application fiber, and focusing optics arranged in the laser light beam path and coupling a part of the laser light output by the laser at the light output location through the light entry cross section of the surgical laser light application fiber into the surgical laser light application fiber. The focusing optics focus the part of the laser light with a full convergence angle of not more than 2° onto the light entry cross section. Further, the focusing optics couple the part of the laser light into the surgical laser light application fiber at an angle between the mean laser light propagation direction of the laser light and the fiber axis, wherein the angle is either a fixed angle in a range from 2° to 10°, or adjustable within a range from 0° to at least 5°.

Other features and advantages of the present invention will become apparent to one with skill in the art upon examination of the following drawings and the detailed description. It is intended that all such additional features and advantages be included herein within the scope of the present invention, as defined by the claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 illustrates the function of a first embodiment of the device of the present disclosure and of the laser apparatus of the present disclosure with adjustable angle between a mean laser light propagation direction of the laser light coupled into an application fiber and the fiber axis at an adjusted angle of 0°.

FIG. 2 illustrates a function of the second embodiment of the device and the laser apparatus of the present disclosure with an angle of 7° between the mean laser light propagation direction of the laser light coupled into the application fiber and the fiber axis.

FIG. 3 illustrates a practical design of the embodiment of the device of the present disclosure according to FIG. 2.

FIG. 4 shows a design of the embodiment of the laser apparatus and a further design of the device of the present disclosure according to FIG. 2.

FIG. 5 exemplifies a detail of FIG. 4 with regard to a use of about 99% of focused laser light by once again coupling-in Fresnel reflections; and

FIG. 6 exemplifies a further detail of FIG. 4 with regard to detecting a used-up state of an application fiber.

### DETAILED DESCRIPTION

A device of the present disclosure serves for connecting an application fiber for applying laser light for surgical applications, or a surgical laser light application fiber to a laser. The application fiber has a light entry cross section running crosswise to a fiber axis, and the laser outputs the laser light at a light output location with a mean laser light propagation direction. The device comprises a laser port for connecting the laser, a fiber port for connecting the application fiber, and focusing optics arranged with respect to the laser port and the fiber port. The laser port orientates the light output location and the mean laser light propagation direction of the laser light with regard to the device. The fiber port orientates the fiber axis and the light entry cross section of the application fiber with regard to the device. The focusing optics are arranged such that they couple the laser light output at the light output location through the light entry cross section of the application fiber into the application fiber connected to the fiber port and, for this purpose, focusses the part of the laser light with a full convergence angle of not more than 2° into the light entry cross section.

Further, the focusing optics couple the part of the laser light output at the light output location at an angle between its mean laser light propagation direction and the fiber axis in a range from 2° to 10° into the application fiber, or the fiber port is tiltable in such a way that the angle between the mean laser light propagation direction of the laser light coupled into the application fiber and the fiber axis is adjustable within a range from 0° to at least 5°.

That the light entry cross section runs crosswise to the fiber axis of the application fiber may—but does not need to—mean that the fiber axis is normal to the light entry cross section. If the fiber axis is not normal to the light entry cross section, the direction in which the light entry cross section is tilted with respect to the fiber axis, the size of this tilt, and the resulting deflection of the laser light in coupling it into the application fiber have to be considered.

In the device of the present disclosure, it is not essential that the part of the laser light output at the light output location, which is coupled into the application fiber and focused into the light entry cross section by the focusing optic, is reduced as compared to the entire laser light output at the light output location. However, as a rule, not the entire laser light output by the laser is coupled into the application fiber, already because the focusing optics have no transmission of 100% and also because a part of the laser light is reflected at the light entry cross section.

By means of the device of the present disclosure, the laser light is coupled into the application fiber only using a small part of a numerical aperture of the application fiber, which is typically about NA=0.22 or above. Correspondingly, at the location of its desired application, the laser light only exits over a small part of the numerical aperture of the application fiber. In practice, typically not more than a tenth of the numerical aperture of the application fiber is used in coupling-in the laser light through the light entry cross section.

The small full convergence angle of the laser light focused into the light entry cross may be achieved in that the focusing optics comprise a collimating lens and a focusing lens, wherein the focusing lens has focal length which is by a factor of at least 5 longer than that one of the collimating lens. In an embodiment, the focusing lens has focal length which is by a factor of at least 8 and, for example, by a factor of about 10 longer than that one of the collimating lens. If the light output by the laser at the light output location is distributed over a larger full divergence angle, i.e. a larger numerical aperture of the laser, an even larger factor between the focal lengths may be necessary or at least reasonable.

The focusing lens of the focusing optics of the device of the present disclosure may have a focal length of at least 10 cm. In an embodiment, the focal length is in a range between 12 cm and 20 cm. Due to the long focal length of the focusing length, a smaller convergence angle of the laser light focused into the light entry cross section and thus a particularly high concentration of the intensity of the laser light exiting out of the application fiber again is realized at a same full divergence angle of the laser light entering the device.

In a particularly simple embodiment, the focusing optics only have the focusing lens, i.e. a single convex lens.

An aperture stop of the device delimiting the full convergence angle may be assigned to the focusing optic. However, in the use of the device of the present disclosure, light running outside the desired full convergence angle is generally not disturbing as long as it is focused into the light entry cross section of the application fiber. It is the function of the aperture stop that laser light reflected back to the laser port does not damage the laser port.

In an embodiment of the device of the present disclosure, the focusing optics couple the part of the laser light output at the light output location at an angle between its mean laser light propagation direction and the fiber axis in a range from 2° to 10° into the application fiber. Thus, the laser light is coupled into a multimode fiber connected to the fiber port as the application fiber in such a way that a ring mode is excited in the application fiber which is also designated as a donut mode. Coupling the laser light at the angle between its mean laser light propagation direction and the fiber axis of the application fiber can be achieved by at least one of an angle between the fiber axis and the mean laser light propagation direction of the laser light focused into the light entry cross section and by a tilt of the light entry cross section with regard to the fiber axis.

In the ring mode excited in the application fiber, the electro-magnetic field of the laser light concentrates to a hollow cylinder coaxially arranged with regard to the fiber axis. The laser light exiting out of a light exit cross section at the distal end of the application fiber which is orientated normal to the fiber axis is not spread out over a full cone but over a hollow cone in which the area of highest light intensity is delimited both outwards and inwards by a cone envelope. Here, a full angle of the inner cone envelope is by the convergence angle of the laser light focused into the light entry cross section smaller than twice the angel between the mean laser light propagation direction of the laser light and the fiber axis, whereas a full angle of the outer cone envelope is by the convergence angle larger than this double angle. Thus, the intensity of the laser light exiting the application fiber is only distributed over a small part of the numerical aperture of the application fiber, and thus over a small part of the area over which the intensity of laser light would be distributed, that would be coupled into the application fiber over the entire numerical aperture of the application fiber, whose numerical application is typically about NA=0.22, corresponding to a full aperture angle of 25°.

Due to the strong concentration of the laser light to the small volume of the hollow cone, a very high intensity of the laser light exiting out of the application fiber is achieved over this volume, and the spatial distribution of this high intensity, i.e. the hollow cone, seems to be very favorable for comminuting water-containing stones. Anyway, with the aid of the device of the present disclosure, high comminuting rates can be achieved with water-containing stones as it will be further explained later. Presumably, this high comminuting rates are also based on that the ring mode generates a flat-top temperature profile in the respective stone. In this way, the applied light energy is optimally used for the evaporation process of the water in the stone, because the water is vaporized in a maximum possible volume.

That the light energy is used in the device of the present disclosure at a higher efficiency means that less light energy and thus also a lower light power are needed for comminuting certain water containing stones. Thus, the danger for tissues located behind or besides the stones to be comminuted, like for example a bladder wall is already considerably reduced. A further danger reduction arises because the area of the cross section of the hollow cone quickly increases with the distance to the application fiber, and, thus, the intensity of the laser light distributed over this cross section quickly decreases. In a comparatively small distance to the application fiber, the intensity of the exiting laser light drops below a lower intensity threshold below which there is no more danger to the tissues onto which the laser light hits. Thus, the applied light energy only triggers the evaporation process of the water used for comminuting the water-containing stones directly in front of the application fiber. Thus, undesired tissue damages are avoidable much easier than with laser light which exits distributed over a full cone having a small full angle.

The focusing optics may couple the part of the laser light output at the light output location into the application fiber at an angle between the mean laser light propagation direction and the fiber axis in a range from 4° to 9°.

In the alternative embodiment of the device of the present disclosure, the angle between the mean laser light propagation direction of the laser light coupled into the application fiber and the fiber axis is adjustable in order to adjust the cone angle of the hollow cone, over which the laser light exiting out of the application fiber is distributed. The angle may be continuously adjustable by tilting the fiber port. By using different application fibers with different inclinations between their light entry cross sections and their fiber axis, the angle can be adjusted in steps. The angle is adjustable in a range from 0° and at least 5°, and it may be adjustable in a range from 0° and at least 7° or at least 10°. The laser light exiting out of the application fiber may then either be directed at an angle of 0° into a full cone with a very small full cone angle, or with increasing angle into a hollow cone with increasing full cone angle approaching the full aperture angle of the application fiber. Thus, for example, laser light in surgical applications may be used once with a high and strongly localized intensity for cutting soft tissue and once with a lower and spatially distributed intensity for coagulating blood.

The laser port of the device of the present disclosure may be a further fiber port for a laser fiber of the laser configured as a fiber laser, or for an optical fiber of the laser forwarding the laser light.

A dichroic beam splitter which couples pump light out of the laser light output at the output location and towards a beam trap of the device may be arranged in the beam path of the laser light between the laser port and the fiber port. Thus, it is ensured, that only the desired laser light is coupled into the light entry cross section of the application fiber. However, often, it is unimportant whether also pump light out of the laser connected to the laser port gets into the application fiber.

Alternatively or additionally, a beam splitter that couples out a percentage of the laser light output at the output location towards a light power sensor of the device in order to register and monitor the intensity of the laser light output at the output location may be arranged in the beam path of the laser light between the laser port and the fiber port. Optionally, a diffusor or a diffusion disc may be arranged in front of the light power detector in order to stabilize the signal of the light power detector.

A beam trap of the device may be arranged in a reflection direction of the part of the laser light focused into the light entry cross section, in order to capture a proportion of the laser light reflected at the light entry cross section of the application fiber. Alternatively, a reflector which reflects the reflected proportion of the laser light which typically makes up about 4% of the laser light focused into the light entry cross section back towards the light entry cross section may be arranged there. Such a reflector may, for example, comprise a hollow mirror, or a reflector lens and a reflector mirror.

In a reverse direction which runs at an angle to the fiber axis that is by at least 2° smaller or larger than the angle between the mean propagation direction of the laser light focused into the light entry cross section and the fiber axis, a further light power sensor of the device may be arranged. This further light power sensor detects light that exits out of the light entry cross section of the application fiber in the reverse direction and which is thus not within the hollow cone which is even there formed by laser light reflected by a perfect light exit cross section at the distal end of the application fiber. Such light outside the hollow cone is an indication that the light exit cross section is no longer undamaged but contaminated or roughened which indicates a use-up of the application fiber.

Further, a pilot laser of the device may be arranged such that it couples visible pilot laser light with a mean pilot light propagation direction along the fiber axis into the application fiber. This pilot laser light may be used for orientating the laser fiber with regard to a stone to be comminuted or any other object to be subjected to the laser light, if the object is viewed, for example, by means of an endoscope or a camera for the visible pilot laser light. Laser light used for comminuting water-containing stones and also for cutting soft tissue and coagulating blood often has a wavelength in the non-visible infrared range.

A laser apparatus of the present disclosure for applying laser light for surgical applications and particularly for comminuting water containing stones in body cavities comprises a laser which outputs laser light at a light output location with a mean laser light propagation direction, an application fiber for applying the laser light comprising a light entry cross section running crosswise to a fiber axis and focusing optics which couple a part of the laser light output by the laser at the light application location through the light entry cross section of the application fiber into the application fiber and, for this purpose, focuses the part of the laser light at a full convergence angle of not more than 2° into the light entry cross section of the application fiber.

Further, the focusing optics couple the part of the laser light output at the light output location at an angle between its mean laser light propagation direction and the fiber axis in a range from 2° to 10° into the application fiber, or the angle between the mean laser light propagation direction of the laser light coupled into the application fiber and the fiber axis is adjustable in a range from 0° to at least 5°.

The focusing optics may couple the part of the laser light output at the light output location at a fixed angle between its mean laser light propagation direction and the fiber axis in a range from 4° to 9° into the application fiber. Alternatively, the angle between the mean laser light propagation direction of the laser light coupled into the application fiber and the fiber axis may be adjustable in a range from 0° and at least 7° or at least 10°.

In the laser apparatus of the present disclosure, like in the use of the device of the present disclosure, the laser light is coupled into the application fiber over a small partial area of its numerical aperture, only, wherein a ring mode may be excited in the application fiber. This has the result that the laser light exiting the application fiber at its distal end is concentrated to a full cone with a very small cone angle or a hollow cone with just a small difference between the full angles of its delimiting inner and outer cone envelopes.

The laser of the laser apparatus of the present disclosure may be a Thulium laser which outputs the laser light at a wavelength of 1,940 nm that is particularly strongly absorbed by water. Further, the fiber may be a fiber laser which only has a small full divergence angle of the laser light output at the light output location such that the full convergence angle of the laser light focused into light entry cross section of the application of not more than 2% may be kept particularly easily. Particularly with a Thulium fiber laser, the laser light output at the light output location may have a beam diameter of not more than 100 μm, or of not more than 50 μm and even of not more than 15 μm, and a full divergence angle of not more than 20°, or of not more than 15°.

A full divergence angle of not more than 20° corresponds to a numerical aperture of the laser of not more than NA=0.175, and a full divergence angle of not more than 15° corresponds to a numerical aperture of the laser of not more than NA=1.13.

The beam diameter of the laser light at the light output location of the laser is considerably enlarged by the necessarily long focal length of a focusing lens of the focusing optic. To nevertheless remain smaller than the light entry cross section of the application fiber, the beam diameter of the laser light at the light output location of the laser may thus not be too big. Otherwise, laser light gets lost between the laser and the application fiber.

As already mentioned, the application fiber of the laser apparatus of the present disclosure is a multimode fiber. A diameter of the light entry cross section of the multimode fiber may be not larger than 1 mm, or not larger than 0.5 mm. It may also only be about 200 μm. A numerical aperture of the multimode fiber is generally in a range between 0.1 and 0.3, or in a range between 0.20 and 0.25, and thus about NA=0.22, which corresponds to a full aperture angle of 26°. In the device of the present disclosure, this full aperture angle is not completely used. Thus, an application fiber with a smaller aperture is also sufficient.

The focusing optics of the laser apparatus of the present disclosure may particularly be the focusing optics of a device of the present disclosure connecting the application fiber to the laser. This implies that the laser apparatus of the present disclosure may comprise all features of the device of the present disclosure.

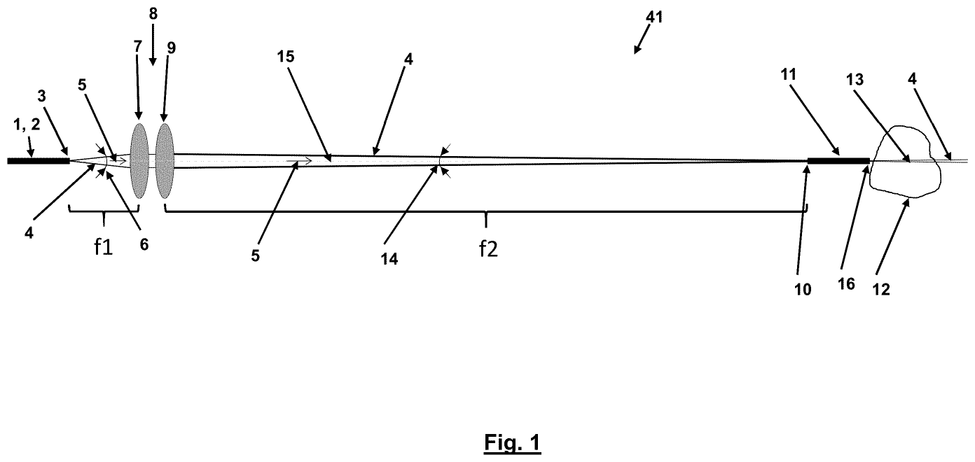

Now referring in greater detail to the drawings, FIG. 1 schematically shows a concept being implemented in the device and the laser apparatus of the present disclosure. A laser 1, which is a Thulium fiber laser 2, outputs laser light 4 at a light output location 3 with a mean laser light propagation direction 5, a full divergence angle 6 of 12.6° corresponding to a numerical aperture NA=0.11, a beam diameter at the light output location 3 of about 10 μm and a wavelength of 1,940 nm. At first, the laser light 4 is collimated with a collimating lens 7 of focusing optics 8 and, then, focused with a focusing length 9 of the focusing optics 8. The collimating lens 7 is arranged at a distance of its focal length f1 to the output location 3. The focusing lens 9 is arranged at a distance of its focal length f2 to a light entry cross section 10 of an application fiber 11 for applying the laser light 4 for surgical applications. In the present case, the light entry cross section 10 runs normal to a fiber axis 15 of the application fiber 11. The application fiber 11 serves for applying the laser light 4, i.e. for directing the laser light 4 onto an object 12, wherein the intensity 4 is concentrated to an application volume 13. The focal length f2 of the focusing lens 9 is ten times longer than the focal length f1 of the collimating lens 7. In this way, a full convergence angle 14 of the laser light 4 focused into the light entry cross section 10 is a tenth of the full divergence angle 6. Thus, it is 1.3° corresponding to a numerical aperture of NA=0.01. The application fiber 11 has a typical numerical aperture of about NA=0.2 or of NA=0.22. Thus, the focused laser light 4 is coupled into the application fiber via the light intensity cross section 10 only using a small part of the numerical aperture of the application fiber 11. Due to the ratio of the focal lengths f1 and f2, a beam diameter of the focused laser light 4 in the area of the light entry cross section 10 is ten times the beam diameter at the light output location 3, i.e. about 100 μm. Thus, the beam diameter of the focused laser light 4 is still clearly smaller than the diameter of the light entry cross section 10 of, for example, 200 μm or 500 μm. The application volume 13 over which the laser light 4 is distributed in the area of the object 12 is small, because the laser light 4 exits at the distal end 16 of the application fiber only distributed over the numerical aperture NA=0.01 over which it has been coupled into the application fiber 11. Correspondingly, the light intensity of the laser light 4 in the application volume 13 is strongly increased as compared to using the full numerical aperture or only half the numerical aperture of the application fiber 11. If the laser light 4, instead of over the numerical aperture NA=0.1, exits out of the application fiber 11 only over the numerical aperture NA=0.01, this corresponds to an increase of the intensity of the laser light 4 in the application volume 3 by a factor of about 100.

In the device according to FIG. 1, an angle between the mean propagation direction 5 of the laser light, which is focused into the light entry cross section 10 of the application fiber 11 and coupled into the laser fiber, and the fiber axis 15 is adjustable but adjusted to 0° so that the fiber axis 15 and the light propagation direction 5 of the focused laser beam 4 coincide.

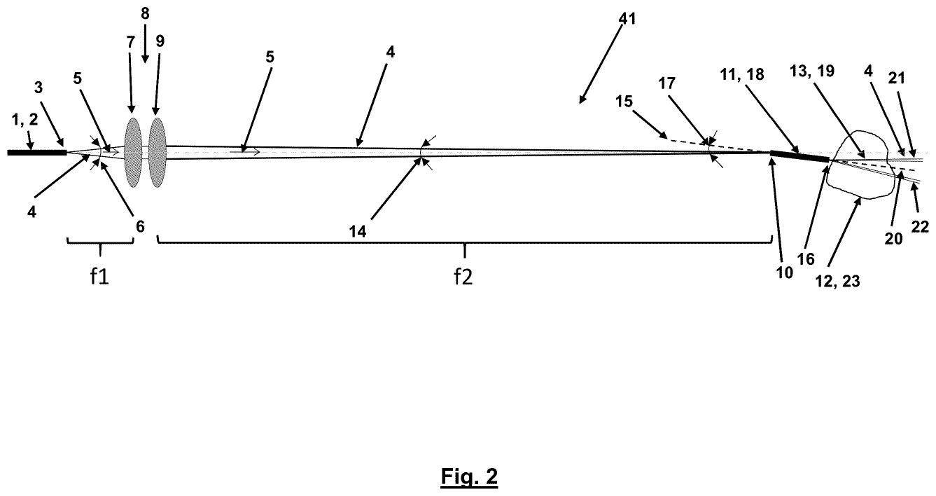

The device according to FIG. 2 differs from that one according to FIG. 1 in that the fiber axis 15 and the light propagation direction 5 of the focused laser beam 4 do not coincide but the mean propagation direction 5 of the laser light 4 focused into the light entry cross section 10 of the application fiber 11 and coupled into the laser fiber is at an angle 17 of 7° to the fiber axis 15. This results in that a ring mode which is also designated as a donut mode is excited in the application fiber 11 made as a multimode fiber 18. For this reason, the laser light 5 in the area of the object 12 exits into the application volume 13 in a shape of a hollow cone 19. A cone axis 20 of the hollow cone 19 is coaxial to the fiber axis 15 at the distal end 16 of the application fiber 11. The hollow cone 19 is delimited by an outer cone envelope 21 and an inner cone envelope 22. A full angle of the outer cone envelope 21 is equal to twice the angle 17 plus half the convergence angle 14 whereas the full angle of the inner cone envelope 22 is equal to twice the angle 17 minus half the convergence angle 14. In the embodiment according to FIG. 2, the laser light 4 is concentrated in the application volume 13 to a lower intensity than in the embodiment according to FIG. 1. However, the shape of the application volume 13 in the shape of the hollow cone 19 proves to be very effective in, for example, comminuting objects 12 in the form of water-containing stones 23. Compared to a full angle corresponding to the full numerical aperture of the application fiber 11 of NA=0.22, the volume of the hollow cone 19 is by about a factor 10 smaller. Compared to laser light from a Holmium:YAG laser of 2,100 nm wavelength, there is also a higher absorption by water which reduces the heated up volume by a further factor of 2. That means that a peak power of a pulse of the laser light, with laser light 4 from the Thulium fiber laser 2, is distributed over a twenty times smaller effective application volume 13 than in case of a Holmium:YAG Laser. Thus, the same light intensity is achieved with a very much smaller pulse energy.

Obviously, there are considerable further advantages due to the shape of the hollow cone 19 of the application volume 13. In any case, a comminuting rate of artificial kidney stones in form of so called Bego stones of 4.5 mg/s is achieved in pulverizing. This is clearly more than previously achieved in pulverizing by means of a Thulium fiber laser with a maximum of 3 mg/sec. The arrangement according to FIG. 2 is also suitable for fragmenting kidney stones and, with 7.5 mg/sec, achieves an even better comminuting rate than the Holmium:YAG Laser which is used for this purpose up to now with 6.6 mg/sec. These figures are based on Thulium fiber lasers of a same peak power and a HOLMIUM:YAG Laser with a twenty times higher peak power than that one of the Thulium-fiber lasers.

Because the high comminuting rate in the device of the present disclosure is achieved with a comparatively small light power of the laser light employed, the danger of unwanted tissue damages in the surroundings of the to be comminuted water-containing stones within body cavities is already considerably reduced. A further reduction of the endangering potential results from that the intensity of the laser light quickly decreases with increasing cross section of the hollow cone.

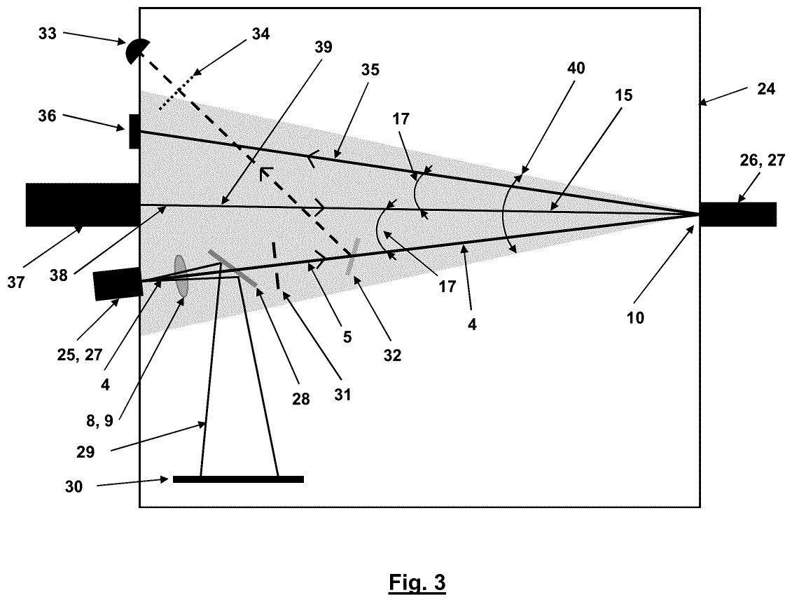

FIG. 3 illustrates a device 24 of the present disclosure for connecting the application fiber 11 to the laser 1 in the arrangement according to FIG. 2. A laser port 25 is provided for the laser 1 or the Thulium fiber laser 2, respectively, and a fiber port 26 is provided for the application fiber 11. Here, the fiber port 26 may be tiltable to vary the angle 17. Both the laser port 25 and the fiber port 26 may be a plug connector 27 for a fiber plug not separately depicted here in which the respective optical fiber is glued-in or fixed otherwise. The focusing optics 8 only comprise the focusing lens 9, here, i.e. the laser light 4 entering into the device 24 at the light output location 32 is not at first collimated and then focused into the light entry cross section 10, but both actions occur in one step. A dichroic beam splitter 28 arranged between the laser port 25 and the fiber port 25 couples pump light 29, which together with the laser light 4 enters into the device 24, towards a beam trap 30. An aperture stop 31 delimits the beam diameter of the focused laser light 4. A beam splitter 32 couples-out a percentage of the laser light 4 entering in to the device 24 towards a light power detector 33, a diffusion disc 34 being arranged in front of the light power detector 33 in order to stabilizing the signal of the light power detector 33 with regard to, for example, the formation of speckle patterns. At the light intensity cross section 10, the application fiber 11 reflects a part of the focused laser beam 4 of about 4% in a reflection direction 35 which is here symmetric to the mean laser light propagation direction 5 of the focused laser beam 4 with respect to the fiber axis 15. In the embodiment according to FIG. 3, this reflected laser light gets into a beam trap 36. A pilot laser 37 is arranged in such a way that it couples visible pilot laser light 38 with a mean pilot light propagation direction 39 along the fiber axis 15 into the application fiber 11 connected to the fiber port 26. Further, a full aperture angle 40 of the application fiber 11, which corresponds to a numerical aperture NA=0.22, is indicated in FIG. 3. This makes clear how little of the full numerical aperture of the application fiber 11 is used in coupling the focused laser light into the light entry cross section 10 according to the present disclosure.

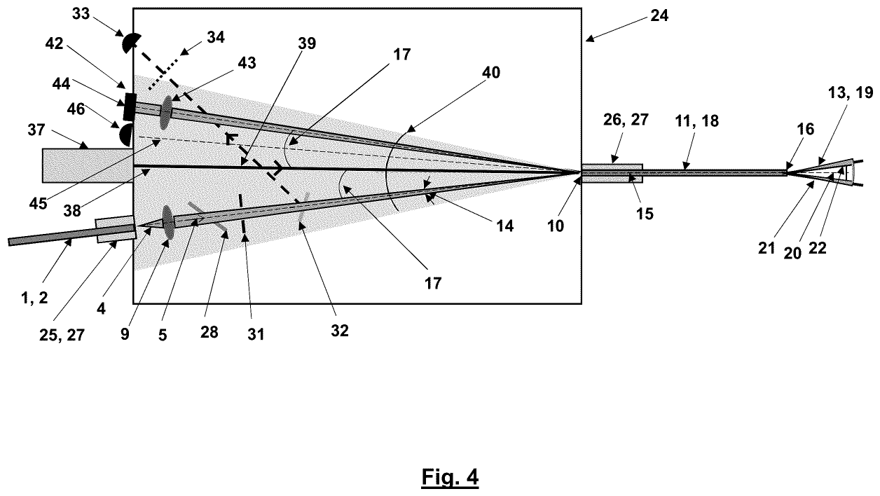

FIG. 4 shows an embodiment of the device 24, which is modified as compared to FIG. 3, as a part of a laser apparatus 41 of the present disclosure. Here, the device 24 comprises the following modifications as compared to FIG. 3. Instead of the beam trap 36, a reflector 42 is provided for the light 4 reflected in the reflection direction 35, the reflector 42 reflecting the reflected laser light 4 back into the light entry cross section 10. In the present case, the reflector 42 comprises a reflector lens 43 and a reflector mirror. Further, a further light power detector 46 is arranged in a reverse direction 45 that this oriented at another angle than the angle 17 to the fiber axis 15.

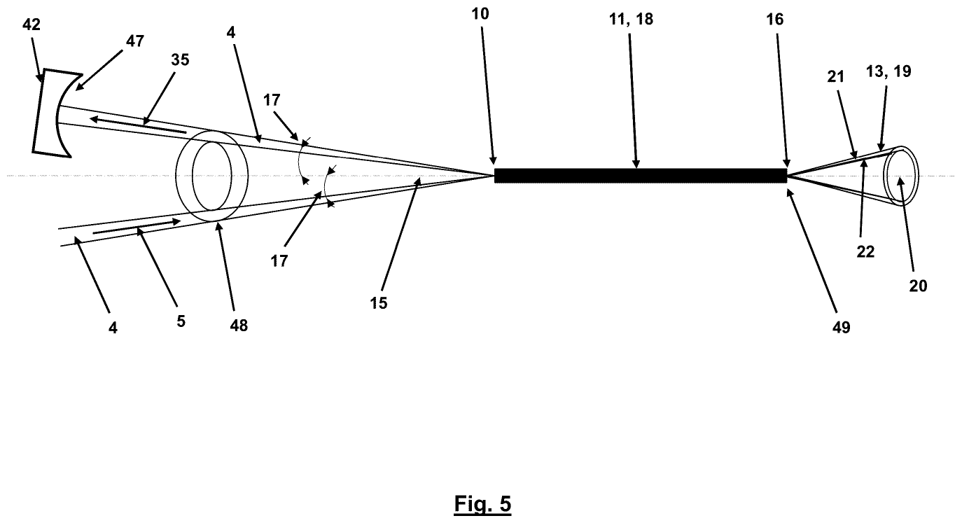

FIG. 5 explains the function of the reflector 42 according to FIG. 4 which is, in the present case, designed as a hollow mirror 47. Laser light 4 reflected by the application fiber 11 at the light entry cross section 10 is reflected by the reflector 42 once again at the angle 17 to the fiber axis 15 into the light entry cross section 10. Thus, 99% of the laser light 4 focused in the light entry cross section 10 are actually coupled into the application fiber 11. Further, FIG. 5 indicates that an image 48 of a reverse reflection at a light exit cross section 49 at the distal end 16 of the application fiber 11 is ring or donut shaped as long as the light exit cross section 49 of the application fiber 11 is clean and smooth as well as normal to the fiber axis 15.

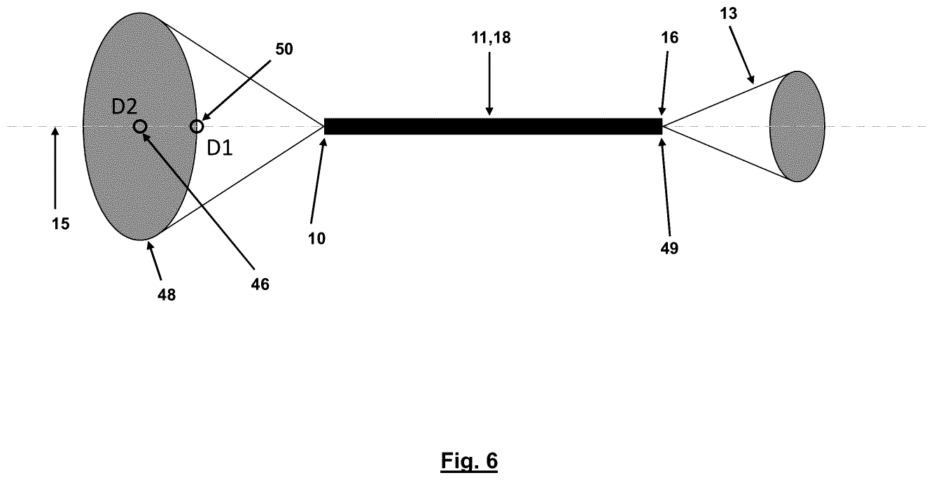

This fact is utilized by the light power detector 46 delineated in FIG. 4. If, according to FIG. 6, the image 48 is no ring but a circular disk, this points to a roughening or another impairment of the light exit cross section 49 of the application fiber 11 at the distal end 16. This roughening also results in that the application volume 13 no longer has the shape of the hollow cone 19 but gets full cone shaped. Even more securely than solely from the signal of the light power detector 46 close to the fiber axis 15, the roughening of the light exit cross section 49 can be detected by the difference of the signal of the light power sensor 46 to a further light power sensor 50 not depicted in FIG. 4, which is arranged in the area of the ring-shaped image 48 of the back reflection according to FIG. 5. In the ring-shaped image 48, the difference of the signals of the two light power sensors 46 and 50 is maximum. With increasing roughening of the light exit cross section 49 this difference decreases. If it drops below a certain value, it is time to replace the application fiber 11.

Many variations and modifications may be made to the preferred embodiments of the invention without departing substantially from the spirit and principles of the invention. All such modifications and variations are intended to be included herein within the scope of the present invention, as defined by the following claims.

I claim:

1. A device for connecting a surgical laser light application fiber to a laser, the surgical laser light application fiber having a fiber axis and a light entry cross section running crosswise to the fiber axis, and the laser outputting laser light at a light output location in a mean laser light propagation direction, the device comprising:
a laser port for connecting the laser to the device, the laser port orientating the light output location and the mean laser light propagation direction of the laser light with regard to the device,
a fiber port for connecting the surgical laser light application fiber to the device, the fiber port orientating the fiber axis and the light entry cross section of the surgical laser light application fiber with regard to the device,
a laser light beam path extending between the laser port and the fiber port, and
focusing optics arranged in the laser light beam path and coupling a part of the laser light output by the laser connected to the laser port at the light output location through the light entry cross section of the surgical laser light application fiber into the surgical laser light application fiber connected to the fiber port,
wherein the focusing optics focus the part of the laser light with a full convergence angle of not more than 2° onto the light entry cross section, and
wherein the focusing optics couple the part of the laser light into the surgical laser light application fiber at an angle between the mean laser light propagation direction of the laser light and the fiber axis, wherein:
the angle is a fixed angle in a range from 2° to 10°, or
the fiber port is tiltable with regard to the focusing optics such that the angle is adjustable within a range from 0° to at least 5°.

2. The device of claim 1, wherein the focusing optics include a collimating lens and a focusing lens, wherein the focusing lens has a focal length which is by a factor of at least 5 longer than that one of the collimating lens.

3. The device of claim 1, wherein the focusing optics comprise a focusing lens having a focal length of at least 10 cm.

4. The device of claim 1, wherein the fixed angle is in a range from 4° to 9°.

5. The device of claim 1, wherein the fiber port is tiltable such that the angle b is adjustable in a range from 0° to at least 7°.

6. The device of claim 1, further comprising a dichroic beam splitter arranged in the laser light beam path and a beam trap, the dichroic beam splitter coupling-out pump light away from the laser light output at the light output location towards the beam trap.

7. The device of claim 1, further comprising a beam splitter arranged in the laser light beam path and a light power detector, the beam splitter coupling-out a percentage of the laser light output at the light output location towards the light power detector.

8. The device of claim 7, further comprising a diffusor or a diffusion disc arranged in front of the light power detector.

9. The device of claim 1, further comprising:
a beam trap, or
a reflector
arranged in a reflection direction of the part of the laser light focused onto the light entry cross section.

10. The device of claim 9, wherein the reflector comprises:
a hollow mirror, or
a reflector lens and a reflector mirror.

11. The device of claim 1, further comprising a further light power detector arranged in a reverse direction which runs at a further angle to the fiber axis that is by at least 2° smaller or larger than the angle between the mean laser light propagation direction of the laser light focused onto the light entry cross section and the fiber axis.

12. The device of claim 1, further comprising a pilot laser coupling visible pilot laser light with a mean pilot light propagation direction on the fiber axis into the surgical laser light application fiber.

13. A laser apparatus for surgical applications, comprising:
a laser which outputs laser light with a mean laser light propagation direction at a light output location,
a surgical laser light application fiber for applying the laser light, the surgical laser light application fiber having a fiber axis and a light entry cross section running perpendicular to the fiber axis,
a laser light beam path extending between the laser and the surgical laser light application fiber, and
focusing optics arranged in the laser light beam path and coupling a part of the laser light output by the laser at the light output location through the light entry cross section of the surgical laser light application fiber into the surgical laser light application fiber,
wherein the focusing optics focus the part of the laser light with a full convergence angle of not more than 2° onto the light entry cross section, and
wherein the focusing optics couple the part of the laser light into the surgical laser light application fiber at an angle between the mean laser light propagation direction of the laser light and the fiber axis, wherein the angle is:
a fixed angle in a range from 2° to 10°, or
adjustable within a range from 0° to at least 5°.

14. The laser apparatus of claim 13, wherein the fixed angle is in a range from 4° to 9°.

15. The laser apparatus of claim 13, wherein the fiber port is tiltable with regard to the focusing optics such that the angle is adjustable within a range from 0° to at least 7°.

16. The laser apparatus of claim 13, wherein the laser is at least one of:
a thulium laser, or
a fiber laser in which the laser light output at the light output location has a beam diameter of not more than 50 μm and a full divergence angle of not more than 20°.

17. The laser apparatus of claim 16, wherein the laser light output at the light output location has at least one of:
a beam diameter of not more than 15 μm, or
a full divergence angle of not more than 15°.

18. The laser apparatus of claim 13, wherein the surgical laser light application fiber is a multimode fiber, wherein a diameter of the light entry cross section is not larger than 1 mm, and wherein a numerical aperture of the multimode fiber is in a range between 0.1 and 0.3.

19. The laser apparatus of claim 18, wherein the diameter of the light entry cross section is not larger than 0.5 mm, and wherein the numerical aperture of the multimode fiber is in a range between 0.20 and 0.25.

20. The laser apparatus of claim 13, further comprising a device for connecting the surgical laser light application fiber to the laser, the device comprising:
a laser port for connecting the laser to the device, the laser port orientating the light output location and the mean laser light propagation direction of the laser light with regard to the device,
a fiber port for connecting the surgical laser light application fiber to the device, the fiber port orientating the fiber axis and the light entry cross section of the surgical laser light application fiber with regard to the device,
the laser light beam path extending between the laser port and the fiber port, and
the focusing optics.
